(19) United States
(12) Patent Application Publication
(10) Pub. No.: US 2023/0329999 A1
(43) Pub. Date: Oct. 19, 2023

(54) PASTE-TYPE COSMETIC CONTAINING NATURAL MOISTURIZING FACTOR

(71) Applicant: COSMED PHARMACEUTICAL CO., LTD., Kyoto-city, Kyoto (JP)

(72) Inventors: Ying-shu Quan, Kyoto-city, Kyoto (JP); Hiroshi Tanaka, Kyoto-city, Kyoto (JP); Fumio Kamiyama, Kyoto-city, Kyoto (JP)

(21) Appl. No.: 17/905,381

(22) PCT Filed: Mar. 4, 2021
(86) PCT No.: PCT/JP2021/008349

§ 371 Date: Aug. 31, 2022

(30) Foreign Application Priority Data
Mar. 11, 2020 (JP) 2020-042415

(51) Int. Cl. A61K 8/73; A61K 8/02; A61K 8/49; A61Q 19/08; A61M 37/00

(52) U.S. Cl. CPC A61K 8/735; A61K 8/0204; A61K 8/4913; A61Q 19/08; A61M 37/0015; A61K 2800/75; A61K 2800/546; A61K 2800/91; A61K 2800/87; A61M 2037/0053

(57) ABSTRACT

Provided is a cosmetic composition that is used for imparting corneum improving effect and exhibits reduced skin irritation. A microneedle type cosmetic composition containing: 30 to 100 mass % of a water-soluble or water-swellable polymer compound; and one or more types of natural moisturizing factors, wherein when the microneedle type cosmetic composition contains 100 mass % of the water-soluble or water-swellable polymer compound, the water-soluble or water-swellable polymer compound is the natural moisturizing factors; and a method for producing the microneedle type cosmetic composition, including: preparing an aqueous solution of a water-soluble or water-swellable polymer compound, and mixing the aqueous solution with one or more types of natural moisturizing factors to produce a cosmetic raw material liquid.

### TECHNICAL FIELD

The present invention relates to a natural moisturizing factor-containing affixing type cosmetic. Specifically, the present invention relates to the technical field of microneedles containing, as a main component, a natural moisturizing factor as a valuable component.

### BACKGROUND ART

The most important role of the skin is to serve as a defense barrier that protects the skin from bacterial invasion and damage from the outside and to prevent loss of heat and moisture from the inside. The skin is composed of a plurality of layers, and among them, the outermost layer is called epidermis. The skin is classified into three parts of the epidermis, dermis, and subcutaneous fat layer under the epidermis.

The epidermis is further subdivided into a stratum corneum, a stratum granulosum, a stratum spinosum, and a stratum basale, but the epidermal cells generated in the stratum basale ascend to the stratum granulosum over 14 days, and keratinization starts. The stratum corneum is composed of differentiated epidermal cells (corneocytes) and intercellular lipids (skin lipid) filling between the epidermal cells. The stratum corneum have a defense function to prevent substances in the body from going outside and protect the human body from external physical, chemical, and biological stimuli.

The corneocytes are linked by a protein called desmosome, and the desmosome degrades as it goes up to the upper part of the stratum corneum, and the cohesive force between the corneocytes becomes weak, and finally, the corneocytes are separated from the skin. For normal skin, the stratum corneum is composed of 15 to 20 layers, and it takes 15 to 20 days for these layers to be completely separated. However, the thicknesses of the epidermis, dermis, and subcutaneous tissue, which are components constituting the skin, decrease with aging. As a result, the moisture content of the skin rapidly decreases, the skin loses elasticity and wrinkles are formed on the skin due to a change in an extracellular matrix (ECM) component that gives tension to the skin, and separation of the stratum corneum becomes slower than usual and the stratum corneum becomes thicker (overlayering of corneum), leading to loss of skin vitality.

In order to improve skin vitality, various skin care cosmetics, hair care products, and the like are used, but there is a problem that an active ingredient is difficult to sufficiently permeate the skin due to the defense function of the stratum corneum. As a method for reliably supplying a medicinal ingredient to a specific site of the skin surface layer and/or the skin stratum corneum, microneedles have been actively studied. For example, there have been proposed a functional micropile (Patent Document 1) in which piles are provided on a substrate, the piles being made of a carbohydrate that dissolves and disappears in vivo, such as maltose, the piles being a tetragonal prism or a cylinder having a square or circular cross section with one side or a diameter of 0.1 to 100 μm and having a length of 0.5 to 500 μm; and a needle for skin (Patent Document 2) in which a plurality of needles containing a biodegradable material such as polylactic acid or maltose as a component are provided around a central member.

Several techniques are known as microneedles containing cosmetic ingredients (Patent Documents 3 to 5). Patent Document 3 states that cosmetic ingredients such as a whitening ingredient, an anti-wrinkle ingredient, a blood circulation promoting ingredient, a nutritional supplement, an antibacterial agent, and a vitamin are delivered as components of a soluble microneedle array. Patent Document 4 discloses a microneedle sheet containing phosphorylated oligosaccharide calcium and a water-soluble polymer for the purpose of normalizing the metabolism of the skin to improve the barrier function by the tight junction formed in the epidermis. Patent Document 5 discloses a microneedle sheet containing a water-soluble or water-dispersible polymer and at least one type of C-glycoside for the purpose of preventing or treating skin aging, particularly skin aging induced by oxidative stress. Patent Documents 4 and 5 state that the microneedle sheet may optionally contain components such as a moisturizer, an antioxidant, and a whitening agent.

### PRIOR ART DOCUMENT

### Patent Document

Patent Document 1: JP 2003-238347 A
Patent Document 2: JP 2006-346126 A
Patent Document 3: JP 2015-522342 A
Patent Document 4: JP 2017-145226 A
Patent Document 5: JP 2019-206500 A

### SUMMARY OF THE INVENTION

### Problem to be Solved by the Invention

The problem to be solved by the present invention are to solve the above-described problems of the prior art, and it is an object of the present invention to provide a cosmetic composition that is used for imparting corneum improving effect and exhibits reduced skin irritation.

### Means for Solving the Problem

Under such circumstances, the present inventors have conducted studies on a cosmetic composition for activating and moisturizing the stratum corneum, and completed a microneedle type cosmetic composition for improving the stratum corneum, the composition containing a natural moisturizing factor as a main component.

The present invention is as follows.

[1] A microneedle type cosmetic composition containing: 30 to 100 mass % of a water-soluble or water-swellable polymer compound; and one or more types of natural moisturizing factors, wherein when the composition contains 100 mass % of the water-soluble or water-swellable polymer compound, the water-soluble or water-swellable polymer compound is the natural moisturizing factors.

[2] The cosmetic composition according to [1], wherein the water-soluble or water-swellable polymer compound is one or more types of water-soluble polymer compounds selected from the group consisting of hyaluronic acid, PEG-grafted hyaluronic acid and salts thereof, hydroxypropyl cellulose, carboxymethyl cellulose, sodium chondroitin sulfate, polyvinyl alcohol, and proteoglycan, or a water-swellable polymer compound that is a crosslinked product of the water-soluble polymer compound.

[3] The cosmetic composition according to [1] or [2], wherein the natural moisturizing factors are contained in an amount of 0.1 mass % or more based on a total mass of the composition.

[4] The cosmetic composition according to [1] or [2], wherein the natural moisturizing factors are contained in an amount of 8 mass % or more based on a total mass of the composition.

[5] The cosmetic composition according to any one of [1] to [4], wherein a component having a molecular weight of 1,000 or less of the natural moisturizing factors is contained in an amount of 70 mass % or less based on a total mass of the composition.

[6] The cosmetic composition according to any one of [1] to [5], wherein the natural moisturizing factors are present in a dissolved or dispersed state in the microneedle type cosmetic composition.

[7] The cosmetic composition according to any one of [1] to [6], wherein the natural moisturizing factors are one or more types selected from substances including amino acids, metabolites of amino acids, pyrrolidone carboxylic acid and salts thereof, ureas, inorganic ions and inorganic salts, and organic acid salts.

[8] The cosmetic composition according to [7], wherein the ureas are a substance selected from the group consisting of urea, ammonia, uric acid, glucosamine, creatinine, and urocanic acid.

[9] The cosmetic composition according to [7], wherein the ureas are glucosamine, or a combination of glucosamine and one or more types of substances selected from the group consisting of urea, ammonia, uric acid, creatinine, and urocanic acid.

[10] A method for producing a microneedle type cosmetic composition, including:
preparing an aqueous solution of a water-soluble or water-swellable polymer compound, and mixing the one or more types of natural moisturizing factors in aqueous solution to produce a cosmetic raw material liquid;
casting the cosmetic raw material liquid into a mold for a microneedle;
heating the mold to dry the cosmetic raw material liquid; and
taking out a cosmetic composition molded into a microneedle from the mold.

[11] The method for producing a cosmetic composition according to [10], wherein the cosmetic raw material liquid is produced by dissolving or suspending the natural moisturizing factors in a mixed liquid of water and alcohol, water and polyhydric alcohol, or water, alcohol and polyhydric alcohol.

### Effect of the Invention

According to the present invention, the skin barrier function can be restored through the corneum-cortex activating effect of the natural moisturizing factor, and a younger and healthy skin can be maintained.

### BRIEF DESCRIPTION OF THE DRAWINGS

FIG. 1 is a cross-sectional view showing an example of a method for producing a microneedle array of the present invention.

### MODE FOR CARRYING OUT THE INVENTION

The microneedle type cosmetic composition has the following constitution.

### Microneedle Array

Conide-like, pyramidal, or needle-like microneedles having a height of 100 to 400 μm stand on a substrate having a thickness of 10 to 200 μm. In the constituent substance of the microneedle, valuable substances are dispersed in a state of being dissolved or suspended in a base.

The valuable substance contains a natural moisturizing factor as a main component. In the present invention, the natural moisturizing factor plays a role of maintaining the physical properties of the stratum corneum in the skin.

The natural moisturizing factor is composed of many substances, and these substances are classified into six types of components. Hereinafter, six components and substances belonging thereto are listed.

Component 1. Amino acids (substances including betaine, aspartic acid, glutamic acid, leucine, isoleucine, methionine, tryptophan, histidine, phenylalanine, threonine, serine, valine, proline, glycine, alanine, lysine, arginine, ornithine, and the like, but not limited thereto)

Component 2. Metabolites of amino acids (substances including pyruvic acid, 2-oxoglutaric acid, succinyl-CoA, fumaric acid, oxaloacetic acid, acetyl-CoA, acetoacetic acid, and the like, but not limited thereto)

Component 3. Pyrrolidone carboxylic acids (pyrrolidone carboxylic acid and salts thereof)

Component 4. Ureas (substances including urea, ammonia, uric acid, glucosamine, creatinine, urocanic acid, and the like, but not limited thereto)

Component 5. Inorganic ions and inorganic salts (substances including sodium, potassium, calcium, magnesium, sodium phosphate, chloride, and the like, but not limited thereto)

Component 6. Organic acid salts (substance including sodium lactate, sodium citrate, sodium hyaluronate, and the like, but not limited thereto)

From the viewpoint of its effect, the ureas are preferably glucosamine, or a combination of glucosamine and one or more types of substances selected from the group consisting of urea, ammonia, uric acid, creatinine, and urocanic acid.

The natural moisturizing factor may be contained in an amount of 0.1 to 100 mass %, preferably 1 to 95 mass %, more preferably 5 to 92 mass %, still more preferably 11 to 70 mass %, and most preferably 12 to 70 mass % based on the total mass of the microneedle. When the natural moisturizing factor is contained in an amount of less than 0.1 mass %, the effect of the natural moisturizing factor becomes too small. A compound such as sodium hyaluronate (organic acid salt), which itself has a structure forming strength and moisture retaining ability, may be contained in an amount of 100 mass %. When the base such as carboxymethyl cellulose (CMC) has low moisture retaining ability, the content of the natural moisturizing factor is desirably 8 mass % or more, more preferably 11 to 70 mass %, and still more preferably 12 to 70 mass %. Low molecular weight components such as inorganic ions, salts, and amino acids have no structure-forming ability by themselves, and thus it is necessary to provide the strength required as a microneedle by blending with a water-soluble or water-swellable polymer compound.

The cosmetic composition of the present invention can further contain a moisturizer, for example, a polyol such as glycerin, dipropylene glycol, or butylene glycol, as other components.

The cosmetic composition of the present invention can further contain all types of components usable in normal cosmetics as other components, for example, an ultraviolet shielding agent, a neutralizer, a thickener, a fragrance, an antiseptic, an antioxidant, and a dye.

For formation of microneedles, a base for maintaining the needle shape while containing a natural moisturizing factor as a main component in a dissolved or dispersed state and also maintaining the stiffness to withstand application to the skin is essential. As the base, a water-soluble polymer compound and a crosslinked product thereof are suitable. For example, non-crosslinked products such as hyaluronic acid, hydroxypropyl cellulose, sodium chondroitin sulfate, polyvinyl alcohol, proteoglycan, and PEG-grafted hyaluronic acid (for example, compounds described in JP 2017-019791 A) are suitable. When these water-soluble polymer compounds are crosslinked at an appropriate crosslinking density using a crosslinking agent, they are not soluble in water but swellable. In the present invention, these compounds are referred to as water-swellable polymer compounds. Among them, a crosslinked product of hyaluronic acid has been reported in many cases, and can be suitably used in the present invention.

The proportion of the base in the total mass of the microneedle is 30 to 100 mass %, and 30 to 90 mass % is appropriate. When the proportion is less than 30 mass %, the mechanical strength of the microneedle is weakened, and when the proportion is more than 90 mass %, there is a risk that the effectiveness is weakened.

However, when the proportion of the water-soluble or water-swellable polymer compound as the base is 100 mass %, the water-soluble or water-swellable polymer compound is a natural moisturizing factor, specifically, sodium hyaluronate as an organic acid salt, proteoglycan, sodium chondroitin sulfate, crosslinked hyaluronic acid, and the like.

In addition, when a large amount of a low molecular weight component of the natural moisturizing factor is contained, the mechanical strength of the microneedle is weakened. Thus, the proportion of a component having a molecular weight of 1,000 or less of the natural moisturizing factor in the total mass is preferably 70 mass % or less.

### Method for Producing Microneedle Type Cosmetic Composition

The production method of the present invention includes the following steps (1) to (4):
(1) preparing an aqueous solution of a water-soluble or water-swellable polymer compound, and mixing the aqueous solution with one or more types of natural moisturizing factors to produce a cosmetic raw material liquid;
(2) casting the cosmetic raw material liquid into a mold for a microneedle;
(3) heating the mold to dry the cosmetic raw material liquid; and
(4) taking out the cosmetic composition molded into a microneedle from the mold.

The steps (1) to (4) are not particularly limited, and any publicly known step can be used. Examples of the method include a method in which a cosmetic raw material liquid (aqueous solution or suspension) produced in the above step (1) with other components added thereto as necessary is cast into a mold having a microneedle shape, and dried, and a cosmetic composition molded into a microneedle is released and taken out. After releasing from the mold, the cosmetic composition is cut into an arbitrary shape (patch), and the cut product is lined with an adhesive support as necessary, and used.

In the step (1), a cosmetic raw material liquid is preferably produced by dissolving or suspending the natural moisturizing factor in a mixed liquid of water and alcohol, water and polyhydric alcohol, or water, alcohol and polyhydric alcohol.

Here, examples of the alcohol include ethanol and isopropanol, and examples of the polyhydric alcohol include glycerin and propylene glycol. As the combination of the alcohol and the polyhydric alcohol, a combination of ethanol and glycerin, and a combination of ethanol and propylene glycol are preferable. The alcohol and/or the polyhydric alcohol contained in the mixed liquid can be appropriately selected according to the type and content of the natural moisturizing factor. The ratio of water to alcohol and/or polyhydric alcohol can also be appropriately set according to the type and content of the natural moisturizing factor.

The size of the patch (microneedle patch) including a microneedle array is 1 to 50 square cm. When the size of the patch is less than 1 square cm, the effect is limited and the effectiveness is not easily exhibited. When the size of the patch exceeds 50 square cm, a problem in adhesion is likely to occur in covering the body surface. In order to cover a wide body surface, a plurality of microneedle patches each having a size of 50 square cm or less may be used.

In order to stably apply the microneedle patch to the skin and hold the microneedle patch on the skin, the microneedle patch more preferably, but not essentially, have an adhesive support on the back surface thereof. The adhesive support is a support film having an adhesive layer on one side thereof.

The adhesive layer in the present invention is determined in consideration of adhesion to the skin and attachment to the support film. Specifically, a material of the adhesive layer is selected from an acrylic adhesive, a rubber-based adhesive, a silicone-based adhesive, a urethane-based adhesive, and the like. In order to improve the stickiness to the skin, a plasticizer such as isopropyl myristate or isopropyl palmitate, a tackifier, a liquid hydrocarbon, or the like may be added to these adhesives. The thickness of the adhesive layer is desirably 10 μm to 250 μm.

As the support film, a film made of a synthetic polymer is preferable, and specifically, polyvinyl alcohol, polyolefin, polyurethane, polyethylene terephthalate (PET), or the like is used.

### EXAMPLES

Hereinafter, the present invention will be described in more detail by the following Examples. These examples are merely examples for specifically describing the present invention, and the scope of the present invention is not limited to these examples.

### Example 1

### Preparation of Microneedle Array

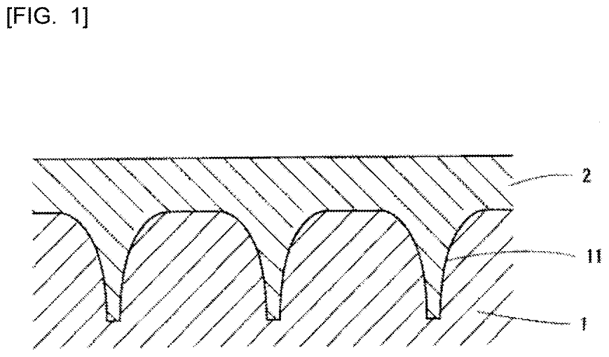

FIG. 1 is a cross-sectional view showing an example of a method for producing a microneedle array of the present invention. In the drawing, reference numeral 1 denotes a mold with a conide type microneedle forming concave portion 11 formed through a process of: forming a conide-type microneedle pattern by a lithography method of irradiating a photosensitive resin with light; and then performing electroforming to transfer the conide-type microneedle pattern. Reference numeral 2 denotes a cosmetic raw material liquid cast into the microneedle forming concave portion 11.

The microneedle forming concave portions 11 are a conide type with a bottom diameter of 0.6 mm, a tip diameter of 0.02 mm, and a depth of 0.25 mm, and are arranged in a lattice pattern at intervals of 0.8 mm.

An aqueous hyaluronic acid solution (cosmetic raw material liquid) prepared by dissolving 20 parts by mass of hyaluronic acid (manufactured by Kikkoman Biochemifa Company, trade name “FCH-SU”, molecular weight: 100,000) and 20 parts by mass of a composite wetting agent (manufactured by Ajinomoto Healthy Supply Co., Inc., trade name PRODEW, composed of a plurality of amino acids <component 1> and pyrrolidone carboxylic acid <component 2>) in 100 parts by mass of water at room temperature was cast into the mold 1, and heated to evaporate moisture in the aqueous hyaluronic acid solution layer. Then, a dried product was released from the mold 1 and punched into an elliptical shape with a size of 7 (minor axis)×50 mm (major axis).

Preparation of Microarray with Protective Adhesive Tape

An elliptical microneedle array with a size of 7 (minor axis)×50 mm (major axis) was set in the central portion of an adhesive tape (HiPAS (registered trademark) 10, adhesive manufactured by CosMED Pharmaceutical Co. Ltd., thickness: 30 μm) with a rectangular (9×56 mm) support (polyethylene film, thickness: 40 μm) having rounded corners to obtain a microneedle array (sheet A) with a protective adhesive tape of the present invention.

### Example 2

### Evaluation of Skin Texture

### [Outline of Test]

The sheet A of Example 1 was applied to the facial part of five subjects and peeled off after five hours. On the next day, the application site on the face of the subject and the adjacent non-application site were observed with a microscope, and the state of the texture was compared with that before use to perform evaluation.

### [Evaluation Results]

### <Evaluation Results of Skin Texture Improving Effect>

In Example 1, regarding the improvement of the skin texture after use of the sheet A, the skin texture was better and improved than before use in all five out of the five subjects. Therefore, it was determined that the sheet A was useful for improving the skin texture.

### Examples 3 to 12

Preparation of Microneedle Array Containing Various Natural Moisturizing Factor Components

Three types of microneedle arrays containing a natural moisturizing factor were prepared in accordance with Example 1. The numbers in the table below are parts by mass in the composition after drying. These compositions were dissolved or suspended in 70 parts of water and 10 parts of an ethanol solution so as to be 20 parts by mass, and dried to form a microneedle array. Note that, in Example 4, hyaluronic acid serves as both the base and the natural moisturizing factor.

TABLE 1

| | | Natural moisturizing factor component (part by mass) | | | | |
|---|---|---|---|---|---|---|
| | Base | Component | Component | Component | Component | Component |
| Examples | (part by mass) | 1 | 3 | 4 | 5 | 6 |
| 3 | Sodium | Alanine | | | | |
| | hyaluronate | (0.05) | | | | |
| | (99.9) | Arginine | | | | |
| | | (0.05) | | | | |
| 4 | | | | | | Sodium |
| | | | | | | hyaluronate |
| | | | | | | (100) |
| 5 | Sodium | Alanine | Pyrrolidone | Urea (2) | | |
| | hyaluronate | (20) | carboxylic | | | |
| | (73) | | acid (5) | | | |
| 6 | Sodium | Proline (1) | Sodium | Glucosamine | NaCl (0.5) | |
| | hyaluronate | | pyrrolidone | (5) | | |
| | (92) | | carboxylate | | | |
| | | | (1) | | | |
| 7 | Sodium PEG- | Arginine | Sodium | Urocanic | KCI (0.5) | Sodium |
| | grafted | (0.1) | pyrrolidone | acid (0.5) | | citrate |
| | hyaluronate | | carboxylate | | | (0.5) |
| | (97.4) | | (1) | | | |
| 8 | Crosslinked | Proline (3) | Sodium | Glucosamine | | |
| | product of | | pyrrolidone | (67) | | |
| | sodium | | carboxylate | | | |
| | hyaluronate | | (1) | | | |
| | (30) | | | | | |
| 9 | Sodium | Alanine (3) | Pyrrolidone | Urea (5) | | |
| | hyaluronate | | carboxylic | Glucosamine | | |
| | (72) | | acid (5) | (15) | | |
| 10 | Hydroxypropyl | Arginine | | Glucosamine | | |
| | cellulose | (0.5) | | (67) | | |
| | (HPC-M) (31) | Alanine | | | | |
| | | (0.5) | | | | |
| | | Proline | | | | |
| | | (1.0) | | | | |
| 11 | Carboxymethyl | | | Urea (5) | | |
| | cellulose (85) | | | Glucosamine | | |
| | | | | (10) | | |
| 12 | Sodium | Arginine | Sodium | | | |
| | chondroitin | (3) | pyrrolidone | | | |
| | sulfate (96) | | carboxylate | | | |
| | | | (1) | | | |

All of the natural moisturizing factor-containing microneedles produced in Examples 1 to 12 had sufficient mechanical strength for skin application.

TABLE 2

| | | Natural moisturizing factor component (part by mass) | | | | |
|---|---|---|---|---|---|---|
| Comparative | Base | Component | Component | Component | Component | Component |
| Example | (part by mass) | 1 | 2 | 3 | 4 | 5 |

| | | | | |
|---|---|---|---|---|
| 1 | Sodium | Arginine | | Glucosamine |
| | hyaluronate (9) | (1) | | (90) |
| 2 | Sodium | | Sodium | |
| | chondroitin | | pyrrolidone | |
| | sulfate (20) | | carboxylate | |
| | | | (80) | |

In Comparative Example 1 and Comparative Example 2, the microneedles are brittle and have poor film properties.

### DESCRIPTION OF REFERENCE SYMBOLS

1 Mold
2 Cosmetic raw material liquid
11 Microneedle forming concave portion

1. A microneedle type cosmetic composition comprising:
30 to 100 mass % of a water-soluble or water-swellable polymer compound; and
one or more types of natural moisturizing factors,
wherein when the composition contains 100 mass % of the water-soluble or water-swellable polymer compound, the water-soluble or water-swellable polymer compound is the natural moisturizing factors.

2. The cosmetic composition according to claim 1, wherein the water-soluble or water-swellable polymer compound is one or more types of water-soluble polymer compounds selected from the group consisting of hyaluronic acid, PEG-grafted-hyaluronic acid and salts thereof, hydroxypropyl cellulose, carboxymethyl cellulose, sodium chondroitin sulfate, polyvinyl alcohol, and proteoglycan, or a water-swellable polymer compound that is a crosslinked product of the water-soluble polymer compound.

3. The cosmetic composition according to claim 1, wherein the natural moisturizing factors are contained in an amount of 0.1 mass % or more based on a total mass of the composition.

4. The cosmetic composition according to claim 1, wherein the natural moisturizing factors are contained in an amount of 8 mass % or more based on a total mass of the composition.

5. The cosmetic composition according to claim 1, wherein a component having a molecular weight of 1,000 or less of the natural moisturizing factors is contained in an amount of 70 mass % or less based on a total mass of the composition.

6. The cosmetic composition according to claim 1, wherein the natural moisturizing factors are present in a dissolved or dispersed state in the microneedle type cosmetic composition.

7. The cosmetic composition according to claim 1, wherein the natural moisturizing factors are one or more types selected from substances including amino acids, metabolites of amino acids, pyrrolidone carboxylic acid and salts thereof, ureas, inorganic ions and inorganic salts, and organic acid salts.

8. The cosmetic composition according to claim 7, wherein the ureas are a substance selected from the group consisting of urea, ammonia, uric acid, glucosamine, creatinine, and urocanic acid.

9. The cosmetic composition according to claim 7, wherein the ureas are glucosamine, or a combination of glucosamine and one or more types of substances selected from the group consisting of urea, ammonia, uric acid, creatinine, and urocanic acid.

10. A method for producing a microneedle type cosmetic composition, comprising:
preparing an aqueous solution of a water-soluble or water-swellable polymer compound, and mixing one or more types of natural moisturizing factors in the aqueous solution to produce a cosmetic raw material liquid;
casting the cosmetic raw material liquid into a mold for a microneedle;
heating the mold to dry the cosmetic raw material liquid; and
taking out a cosmetic composition molded into a microneedle from the mold.

11. The method for producing a microneedle type cosmetic composition according to claim 10, wherein the cosmetic raw material liquid is produced by dissolving or suspending the natural moisturizing factors in a mixed liquid of water and alcohol, water and polyhydric alcohol, or water, alcohol and polyhydric alcohol.
